(19) United States
(12) Patent Application Publication
(10) Pub. No.: US 2011/0245747 A1
(43) Pub. Date: Oct. 6, 2011

(54) Splint for treatment of musculoskeletal injury of the hand

(75) Inventors: Ronit Wollstein, Pittsburgh, PA (US); Thomas Ogden, Pittsburgh, PA (US); Jonathan Pearlman, Pittsburgh, PA (US); Rory A. Cooper, Gibsonia, PA (US); David White, Pittsburgh, PA (US); Miriam Zisook, Glencoe, IL (US)

(73) Assignee: Department of Veterans Affairs Rehabilitation

(21) Appl. No.: 12/929,877

(22) Filed: Feb. 22, 2011

Related U.S. Application Data
(60) Provisional application No. 61/306,656, filed on Feb. 22, 2010.

(51) Int. Cl. A61F 5/00

(52) U.S. Cl. 602/22

(57) ABSTRACT

A splint for treatment of a joint including a generally longitudinal body including first and second portions forming inner and outer splint layers, with the second portion being rollable onto the first portion to form the splint. The outer splint layer may include a longitudinal cavity for insertion of a stay, and/or may include a fluted section for permitting insertion of a stay between the inner and outer splint layers. The longitudinal body may be linear or curved. The first and second portions may include areas having different thicknesses for adding rigidity to the splint structure at a predetermined location. The splint may include a cutout for exposing a predetermined portion of a user's finger. The splint may be made of a flexible material such as rubber, silicone and/or urethane. The splint may include a reduced friction surface layer for minimizing sticking of the splint during donning.

### CROSS-REFERENCE TO RELATED APPLICATION

This application is based on U.S. Provisional Application Ser. No. 61/306,656, filed Feb. 22, 2010, the priority of which is hereby claimed. The contents of this priority application are incorporated herein by reference.

### BACKGROUND OF INVENTION

a. Field of Invention

The invention relates generally to splints and other such devices, and more particularly, to a splint for treatment of musculoskeletal injury of a hand by, for example, providing a stabilizing and/or a flexing torque to a joint of a human finger.

b. Description of Related Art

Contracture of a proximal interphalangeal (PIP) joint of a hand can cause significant functional limitations and pain. Referring to FIGS. 1A-1D, contracture of a PIP joint is illustrated in a human finger 10 which includes vertical and dorsal sides 12, 14, distal, middle and proximal phalanxes 16, 18, 20, and interphalangeal (PIP) joints 22, 24, 26 between the phalanxes. Dupuytren's disease, the most common cause of PIP contractures, affects 5-15% of males over 50 years of age in the United States. In prior studies performed in this area, after a combination of surgery and splinting, only 57% of the patients achieved full extension. There are many surgical options for releasing contractures, but typically without hand therapy and splinting the contractures return. Even with surgery and aggressive splinting, the rate of recovery is low.

There are many different types of known splints for PIP contracture, with none generally having very good patient outcomes. Because of the small size of the joint, splints tend to be bulky and oversized. This can increase the functional limitations of the user because the splints can be more cumbersome than the un-splinted affected fingers. Additionally, many of the splints do not adequately distribute load intended to extend the joint, which results in high-pressure points across the finger, which can lead to skin breakdown, pain and discomfort. As shown in FIG. 1A, the area where complications are most common is dorsal side 14 of the PIP joint. To be effective, a splint must be worn for many hours each day, but this can present serious risks for circulation and skin health.

As shown in FIGS. 2A-2D, existing splint designs have springs and outriggers which need to be set by a therapist and can be easily knocked out of the correct position. Bulky splints can be difficult to fit and improper fit can diminish effectiveness and lead to complications. The splints are also difficult to keep in place and often roll and slip around the finger. For example, gutter splint 40 of FIG. 2A tends to roll around a user's finger and must be custom made. The gutter splint design can also put pressure on sensitive areas of a joint. Oval-8-splint design 42 of FIG. 2B can be difficult to put on, and can put damaging pressure in very small sensitive areas. Further, the Bunnel splint design 44 of FIG. 2C may not provide enough corrective force, and can be bulky and roll around a user's finger, and the R. Knuckle bender splint design 46 of FIG. 2D can be cumbersome in nature from use and functionality standpoints. There are currently no splints available that adequately straighten the joint without health complications even when the patient adheres to the treatment. Because of the same problems that cause health complications and functional limitation, therapists also see low rates of adherence to known regimens. Moreover, even an otherwise very effective splint is only effective if it is worn regularly.

In an exemplary known splint design, U.S. Pat. No. 5,095,897 discloses a neoprene splint which is relative low-profile, but the method of fabrication thereof results in a seam, which protrudes from the finger. While some existing splint designs do not have such a seam, they are nevertheless of some standard tubular or tapered form and are not designed to mimic exactly the shape of the joint that is being addressed.

It would therefore be of benefit to provide a splint design which is not only strong enough to be effective in increasing range of motion of the PIP joint, but also encourages higher rates of adherence by addressing one or more of the aforementioned drawbacks with existing splint designs.

### SUMMARY OF THE INVENTION

The invention overcomes the deficiencies of prior art splint designs by providing a splint which is effective in increasing range of motion of the PIP joint and encourages higher rates of adherence. An exemplary objective of the invention is to provide a splint design which has cavities for battens which can be inserted into the splint after it is donned, and which can provide restoring force to the finger, while offering the ability to adjust the joint restoring force by simply removing or adding stiffer battens. Another exemplary objective of the invention is to provide a splint design that does not include a seam. Another exemplary objective of the invention is to provide a splint that is fabricated in a pre-flexed shape, so that when the splint is donned, it tends to flex/extend the joint (depending on the orientation of the splint) in the desired direction. Yet another exemplary objective of the invention is to provide a splint that is fabricated of low-friction material, and is donned by rolling the splint within itself, rather than sliding it over the finger. In this regard, because the splint material friction is low, it is relatively easy to slide on, reducing the pain compared to sliding or fixing other splints on the finger. Another exemplary objective of the invention is to provide a splint that can be worn without creating pressure sores, with the product thus being more comfortable for patients, and having flexible stiffness.

For the splint design described herein, another exemplary objective of the invention is to provide a splint that provides a stabilizing and/or a flexing torque to the joint(s) of the human finger. In an exemplary embodiment, the splint design disclosed herein can target the proximal interphalangeal (PIP) joint, although other joints could equally be targeted. Due to the sensitive nature of the PIP joint soft-tissue structures, any acute damage can result in a joint contracture, with the splint design described herein being designed to correct the contracture.

To address concerns about pain, discomfort and health, which should encourage a user to wear the splint according to a treatment regimen, another exemplary objective of the invention is to provide a splint that is comfortable and held in place by circumferential tension with the pressure diffused over the whole finger. Other exemplary objectives of the invention are to provide a splint that permits visual monitoring of skin health and circulation, and which preserves function in the hand and also addresses the aesthetic concerns of the user by including a low profile so that the splint does not interfere with activities of daily living. Yet further exemplary objectives of the invention are to provide a splint that straightens the joint by using a flexible comfortable material that is combined with a stabilizing force of between 250-300 grams, and a splint that is form-fitting for a variety of finger sizes and still very easy to don and doff.

In an exemplary embodiment, the invention provides a low-profile splint that conforms to the finger similar to a tight-fitting glove. This is achieved by building the splint from an elastic and resilient material (e.g., urethane or silicon) in the basic shape of a finger. Because the splint mimics the shape of the finger, when donned, it applies near-uniform pressure on the surface of the finger. This reduces the likelihood of pressure sores, which are partially caused by high pressure points on the skin so as to occlude blood flow.

In an exemplary embodiment, the invention provides a splint for treatment of a joint. The splint may include a generally longitudinal body including first and second portions forming inner and outer splint layers, with the second portion being rollable onto the first portion to form the splint.

For the splint described above, the outer splint layer may include one or more generally longitudinal cavities for insertion of a stay for adding rigidity to the splint structure. In an embodiment, the splint may include a plurality of cavities disposed in a random or symmetrical pattern around the splint body. In an embodiment, the outer splint layer may include one or more fluted sections for permitting insertion of a stay between the inner and outer splint layers for adding rigidity to the splint structure. In another embodiment, the generally longitudinal body may be generally linear or curved. In an embodiment, the generally longitudinal body may be curved in a predetermined direction so as to place a predetermined torque on a joint when the splint is donned. In yet another embodiment, the first and/or second portions may include areas having different thicknesses for adding rigidity to the splint structure at a predetermined location. In an embodiment, the splint may further include a crease integrally formed along a length of the splint for facilitating predetermined donning of the splint. In another embodiment, the splint may be used for treatment of contracture of a proximal interphalangeal joint of a hand. In an embodiment, the splint may further include one or more cutouts for exposing a predetermined portion of a user's finger. In yet another embodiment, the splint may be made of a flexible material such as rubber, silicone and/or urethane. In an embodiment, the splint may further include a reduced friction outer surface layer for minimizing sticking of the splint during donning, which in a particular embodiment, may include a fabric, a lubricant and/or a powder layer.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIGS. 1A-1D (similarly shown in Colditz, J C. Efficient Mechanics of PIP Mobilisation Splinting. J Hand Ther [Br] 2000; 5(3): 65-71) show various views of contracture of the proximal interphalangeal (PIP) joint of a hand, showing functional limitation, with FIG. 1A showing a healthy PIP joint, and FIGS. 1B-1D showing PIP joint contracture;

FIGS. 2A-2D are exemplary splints having springs and outriggers, with FIGS. 2A-2D respectively illustrating a gutter splint, an oval-8-splint, a Bunnell splint and a R. Knuckle bender splint;

FIGS. 3A-3D show exemplary embodiments of generally straight splints according to the invention;

FIGS. 4A-4D show an embodiment of a splint with placement of rigid inserts either bilaterally, radially or dorsal and ventrally, an embodiment with thirteen radial cavities, an embodiment with four stays, and an exemplary placement of rigid inserts in a splint;

FIGS. 5A-5G show an embodiment of a generally straight splint according to the invention, and steps for donning the splint;

FIGS. 6A-6E show various views of curved splints according to the invention, with some embodiments including a crease for helping the splint stay on a user's finger;

FIGS. 7A-7B show a mold for manufacturing the generally straight splints according to the invention;

FIGS. 8A-8F show various views of molds for manufacturing the generally curved splints according to the invention;

FIGS. 9-11 show further exemplary embodiments of splints according to the invention.

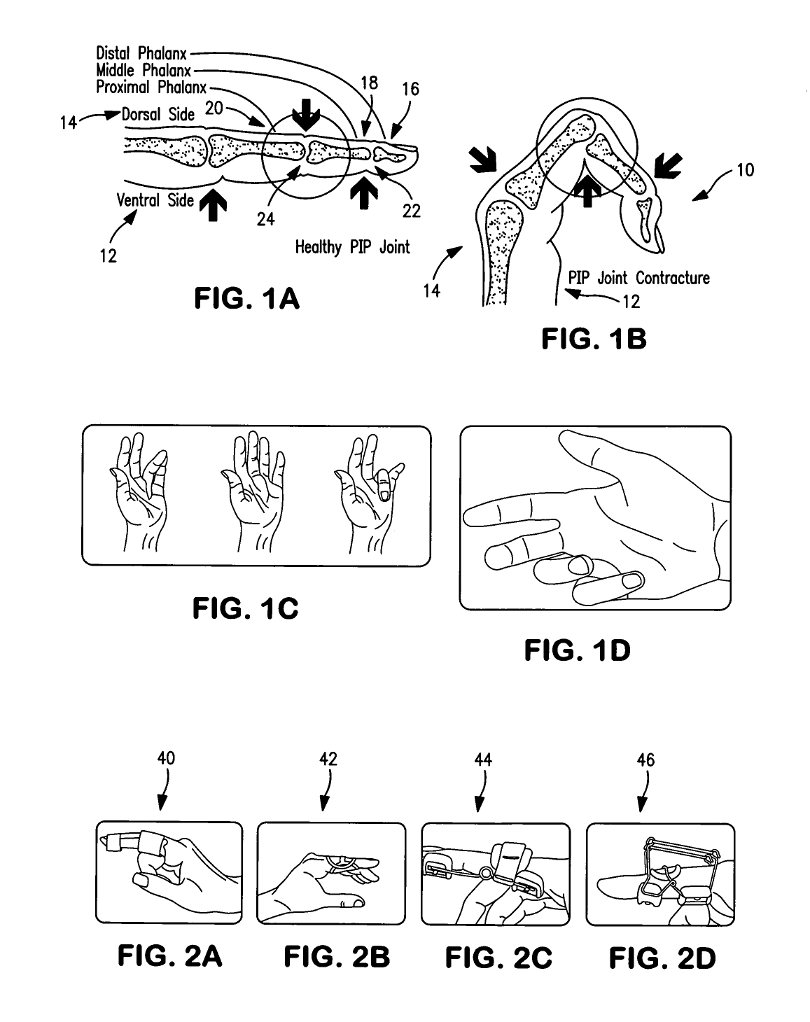

### DETAILED DESCRIPTION OF THE PREFERRED EMBODIMENTS

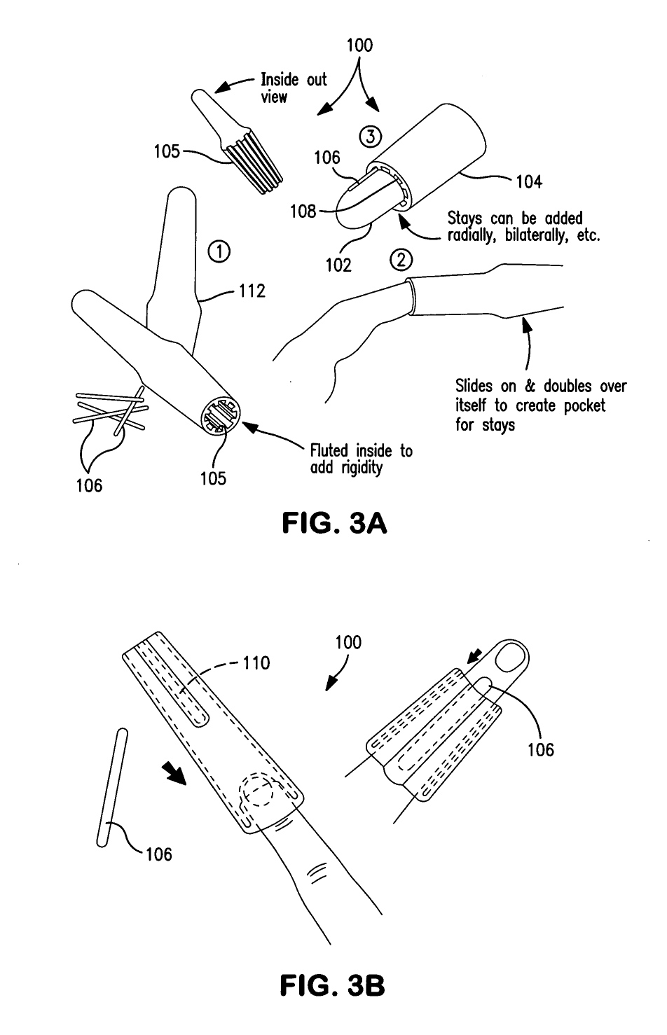

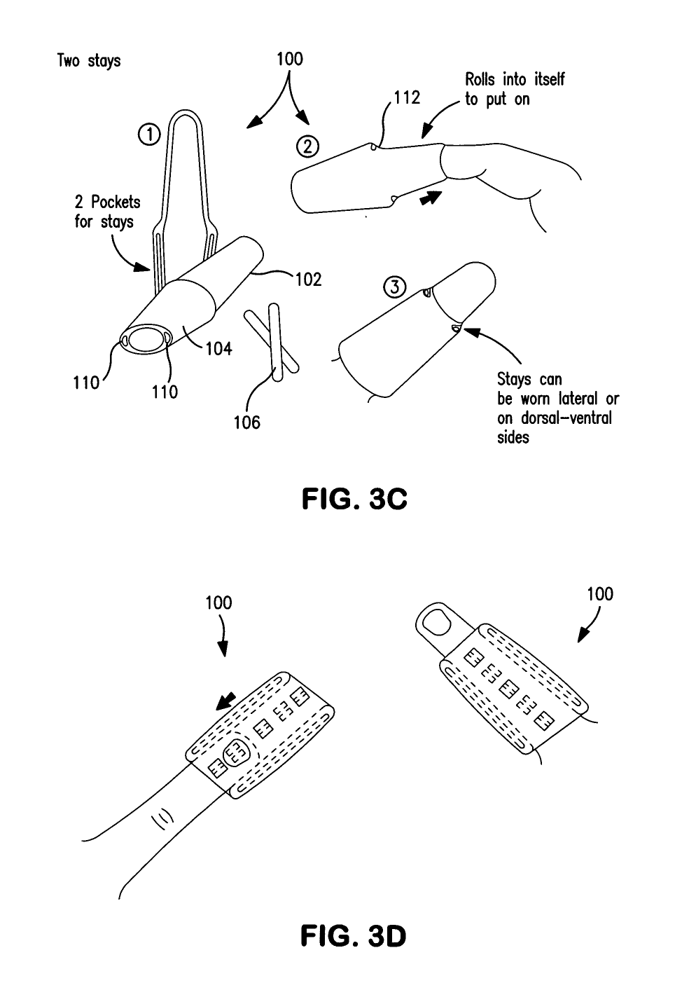

Referring now to the drawings wherein like reference numerals designate corresponding parts throughout the several views, FIGS. 3A-11 are views illustrative of splints according to the invention for treatment of musculoskeletal injury of a hand, and manufacturing and use methods thereof.

Referring to FIGS. 3A-3D, according to an embodiment of the invention, a splint 100 is generally formed of a soft rubber sleeve which may double over itself easily when the finger is inserted so as to alleviate the strain of trying to stretch or roll the splint over the affected joint. Splint 100 may generally include first and second portions forming inner and outer splint layers 102, 104, with outer layer 104 being fluted at 105 in a generally longitudinal direction for adding rigidity to the splint. Additional rigidity may be added by means of stays 106 (e.g. battens) insertable in cavities 108 formed between inner and outer layers 102, 104 for the FIG. 3A embodiment, and within pre-formed cavities 110 for the FIGS. 3B-3D embodiments. As shown in FIG. 3A, an integrated crease 112 may be provided for maintaining the splint in place once donned, and also for guiding a user to don the splint in a predetermined configuration such that the crease generally prevents further movement of outer layer 104.

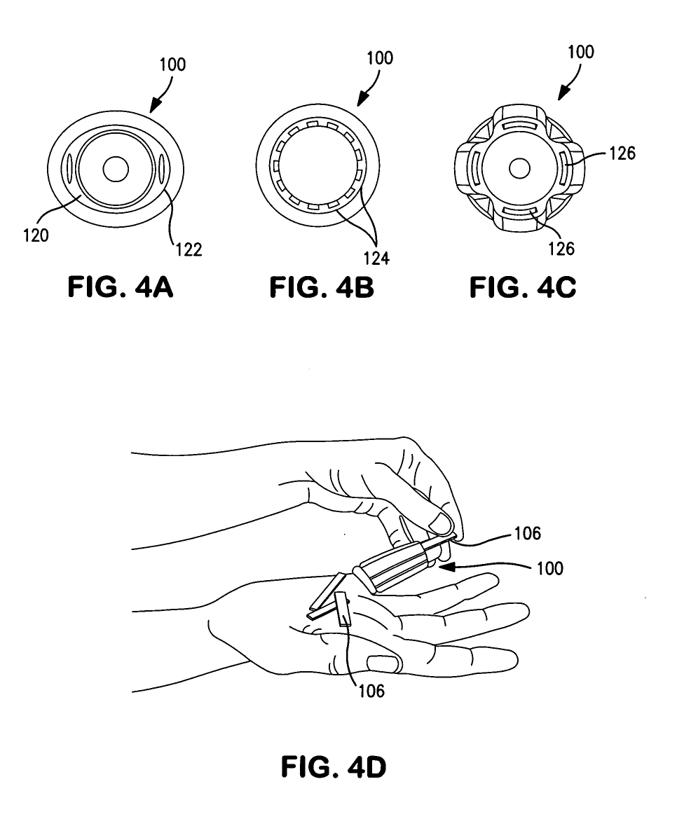

In order to determine where stays may be placed so they would exert enough corrective strength but minimize the pressure in sensitive areas, as shown in FIGS. 4A-4D, stays 106 may be placed either bilaterally, radially or dorsal and ventrally depending on an individual's needs. For example, as shown in FIG. 4A, stays may be placed in lateral or dorsal/ventral cavities 120, 122 for providing support along two opposing directions of movement only. As shown in FIG. 4B, an embodiment with thirteen cavities 124 provides generally uniform bending support in all directions. For the embodiment of FIG. 4C, up to four stays can be placed uniformly around splint 100 within cavities 126. Of course those skilled in the art would appreciate in view of this disclosure that a stay (or stays) may be selectively inserted in one or more cavities for providing support in a desired direction only, regardless of the number of cavities in a splint. The insertion of stays 106 is shown in FIG. 4D, and will discussed in further detail below. The size, rigidity, number, orientation and length of the stays may be adjusted or adjustable so that a therapist can control what angle or rigidity is required for a patient.

The flexibility that allows the splint to be comfortable and form-fitting can present challenges. For example, it is possible for the material to ‘hammock’ or stretch around a resilient insert, defeating the goal of providing a steady extension moment to the joint. To address this concern, by altering the wall thickness in different parts and altering the position of the stays, these problems can be minimized or eliminated. Increasing the wall thickness decreases stretch, such that areas that need to be more rigid can be thicker. This means it can be helpful to increase the wall thickness in the areas around the stays.

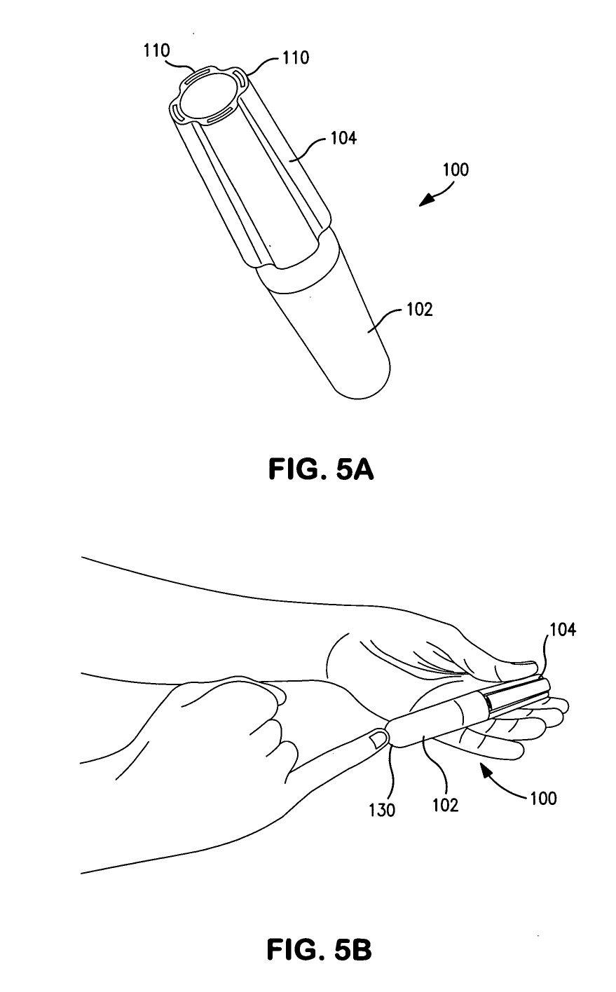

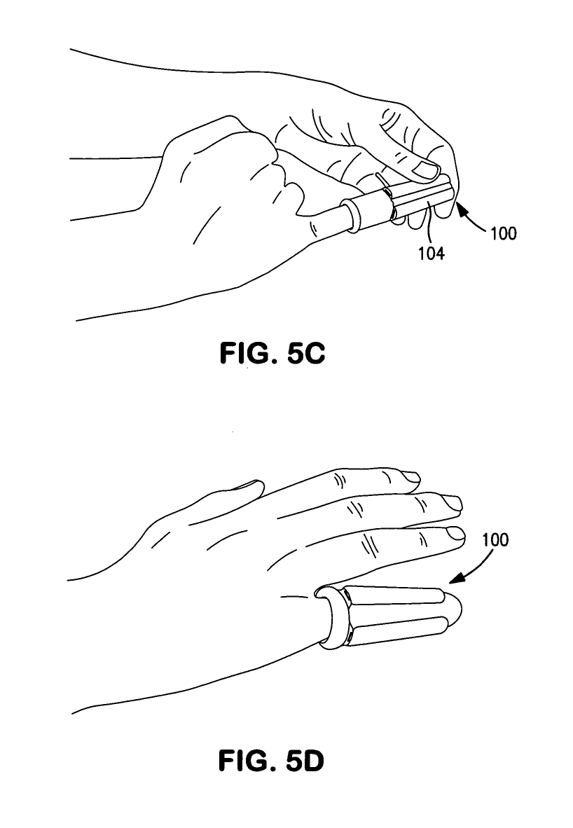

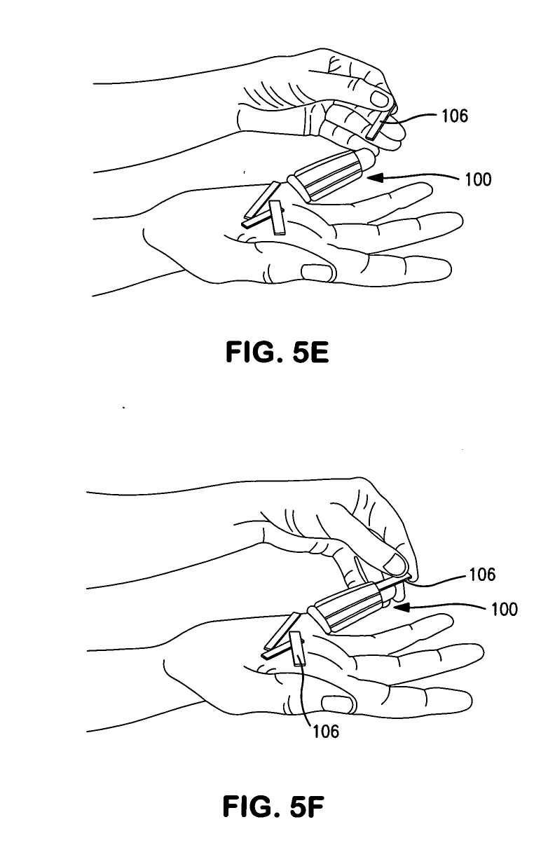

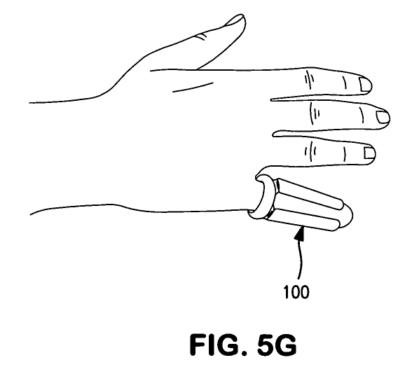

An exemplary method of use of splint 100 according to the invention with now be described in detail with reference to FIGS. 5A-5G.

Referring to FIGS. 5A and 5B, splint 100 may be disposed in its expanded configuration prior to use. As shown in FIGS. 5B and 5C, a user may place end 130 at the end of a finger and move outer layer 104 towards the finger. As shown in FIGS. 5C and 5D, outer layer 104 is fully rolled onto inner layer 102. In this position, as shown in FIG. 5E, stays 106 may be selectively added into the cavities (110; see also embodiments of FIGS. 3B-3D) for adding further rigidity to splint 100.

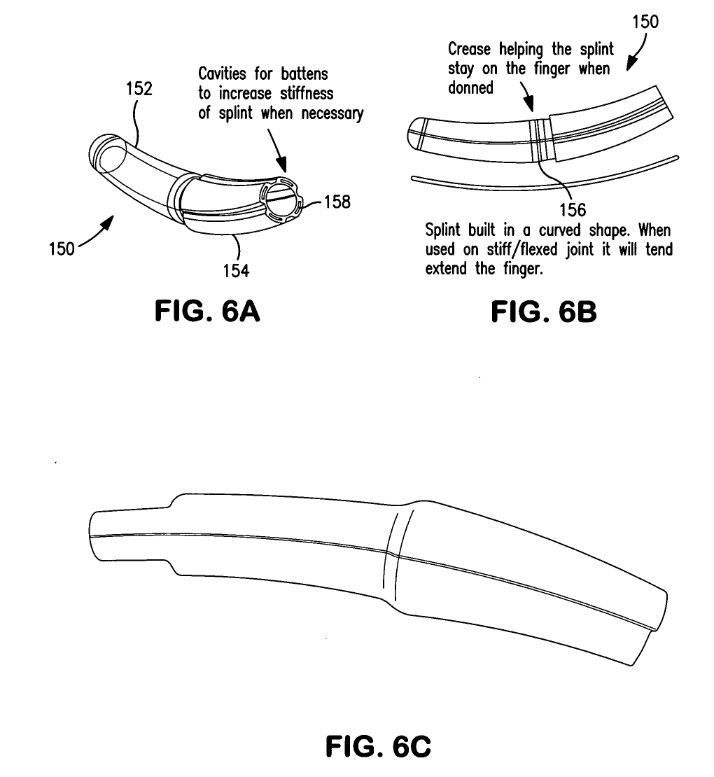

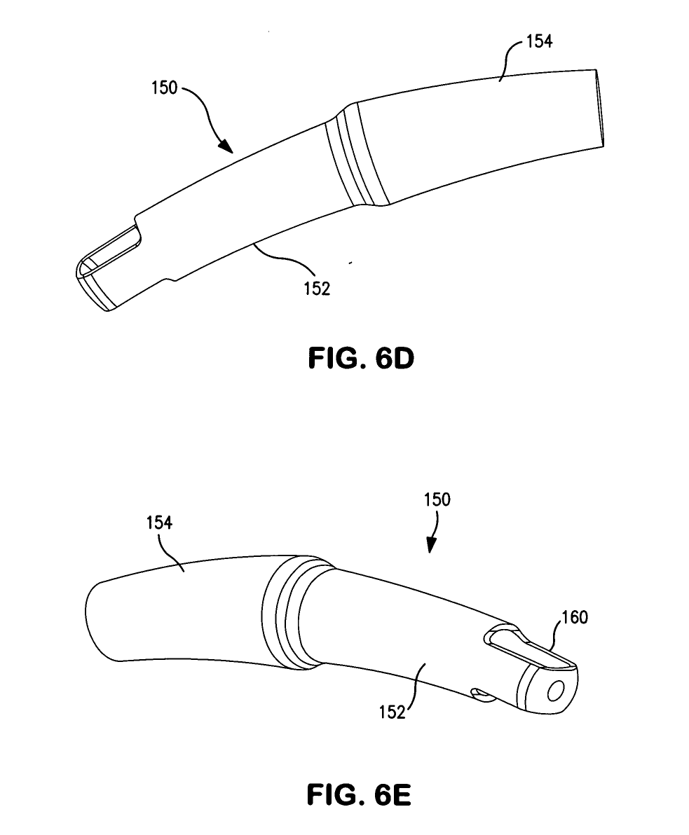

Referring next to FIGS. 6A and 6B, a generally curved splint 150 is illustrated. Curved splint 150 may generally include first and second portions forming inner and outer splint layers 152, 154, with outer layer 154 including cavities in a generally lengthwise direction for adding rigidity to the splint. An integral crease 156 may be provided generally midway along the length of splint 150 for facilitating retention of the splint on a finger when donned. Crease 156 also guides a user to don the splint in a predetermined configuration such that the crease generally prevents further movement of outer layer 154 beyond the fully donned position. As discussed earlier with reference to splint 100, stays 106 may then be inserted into cavities 158. As shown in FIGS. 6D-6E, splint 150 may include a cutout area 160 for exposing the end of a user's finger (e.g. the finger-print and nail areas) to enable adequate ventilation during use as well as allowing a user to monitor the affected finger. Those skilled in the art would appreciate in view of this disclosure that for curved splint 150, in certain situations splint 150 may be manufactured without cavities altogether (see FIGS. 6C-6E) since the curvature of the splint may itself provide an adequate force for maintaining a patient's finger in a required extended direction to help treat the contracture. Thus for curved splint 150, the use of stays actually serves to complement the predetermined restoring torque provided by the curved nature of the splint.

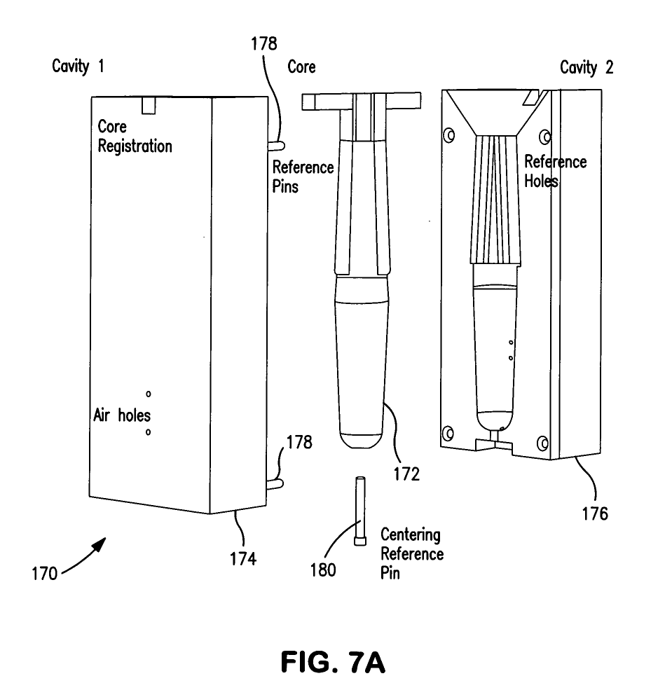

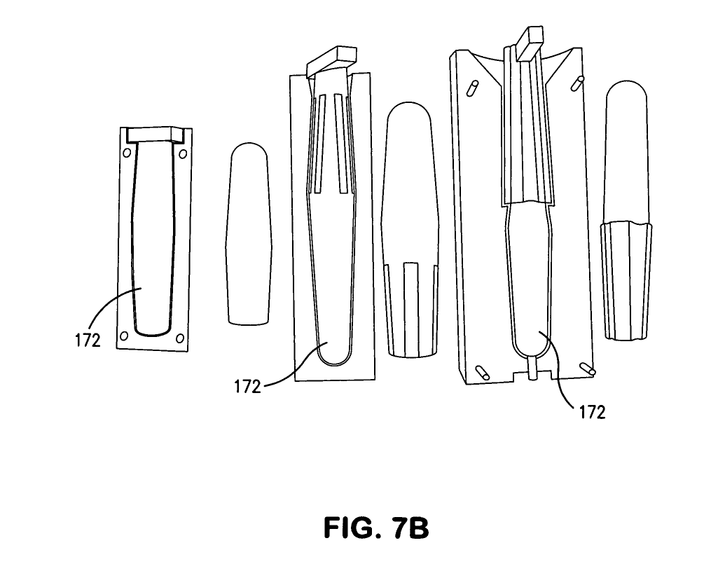

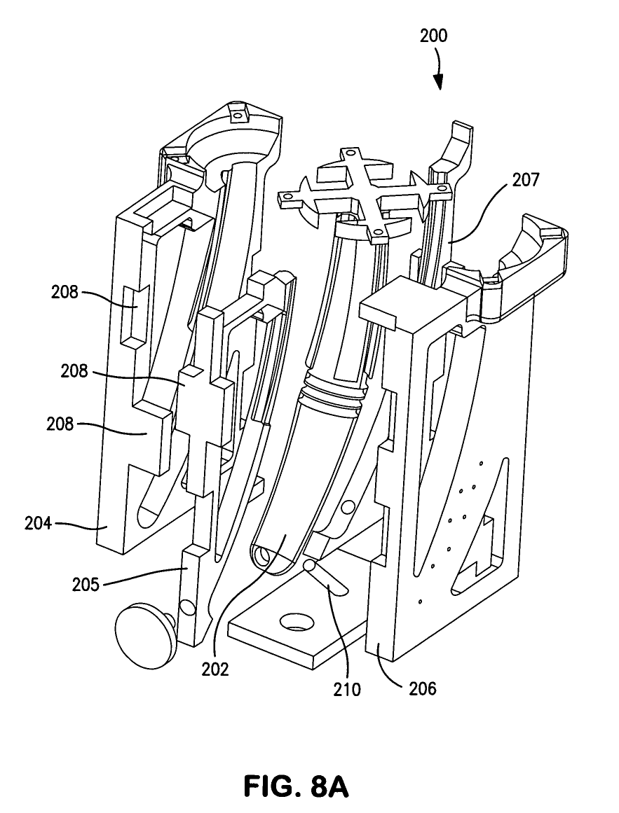

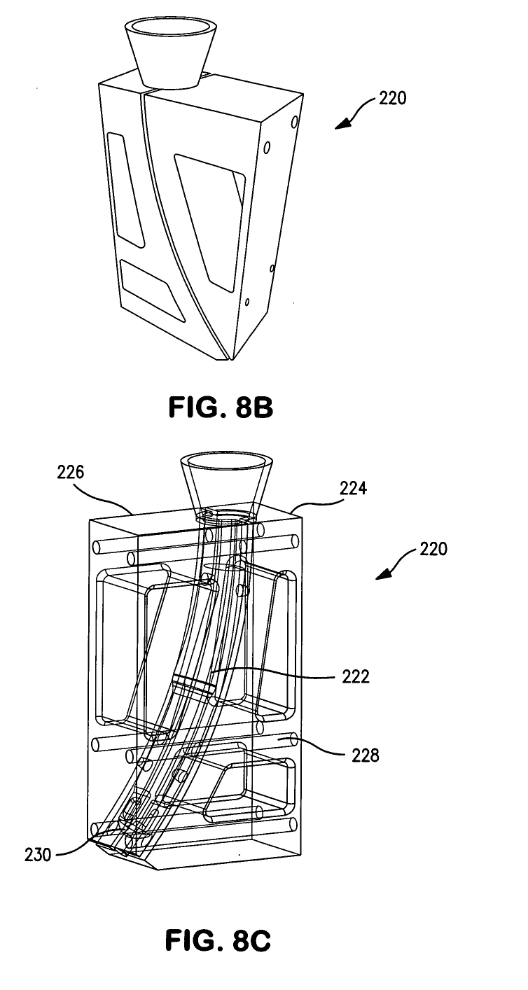

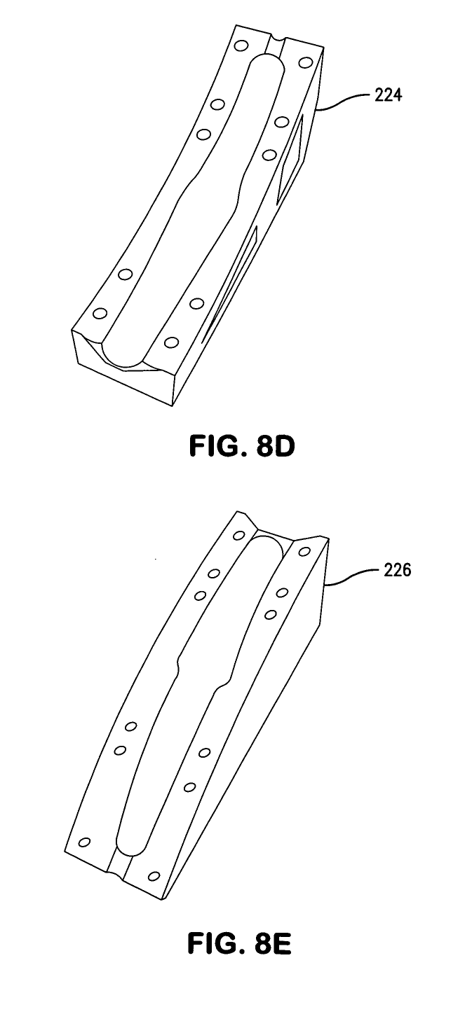

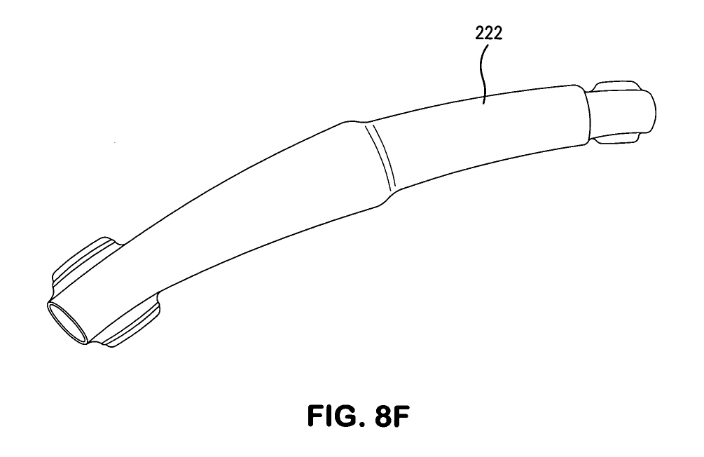

The manufacture of generally straight splint 100 according to the invention will now be described in detail with reference to FIGS. 7A-7B, and generally curved splint 150 with reference to FIGS. 8A-8F.

Referring to FIGS. 7A-7B, splint 100 may be manufactured in a mold 170 including, for example, three parts: a core 172 and two halves 174, 176 including a cavity. Halves 174, 176 may include reference pins 178 for relative alignment thereof. Core 172 may be registered on the top and further include reference pins on the sides and/or through the bottom (e.g. pin 180).

For the mold to pour correctly with the splint wall thickness being relatively small, the core may be aligned through the center and adequate holes may be provided in the bottom and sides for air to escape. Because of the relatively thin walls of splint 100 and the time it takes time for the liquid splint material (e.g. urethane) to pour into the mold, an adequate space may be created in the top of the mold to pour the liquid material into. A funnel of the type shown in FIG. 8B (described with reference to generally curved splint 150) may be used.

Referring to FIG. 8A, splint 150 may be manufactured in a similar manner as splint 100 using a mold 200 including, for example, three parts: a core 202, two side supports 204, 206, and two front/back supports 205, 207. Side supports 204, 206 and front/back supports 205, 207 may include reference protrusions/indentations 208 for relative alignment thereof. Core 202, similar to core 172 of mold 170, may be registered on the top and further include reference pins on the sides and/or through the bottom (e.g. pin 210).

Alternatively, referring to FIGS. 8B-8F, splint 150 may be manufactured in a similar arrangement as mold 170 using a mold 220 including, for example, three parts: a core 222 and two halves to a cavity 224, 226. Halves 224, 226 may include reference pins 228 for relative alignment thereof. Core 222, similar to core 172 of mold 170, may be registered on the top and further include reference pins on the sides and/or through the bottom (e.g. pin 230).

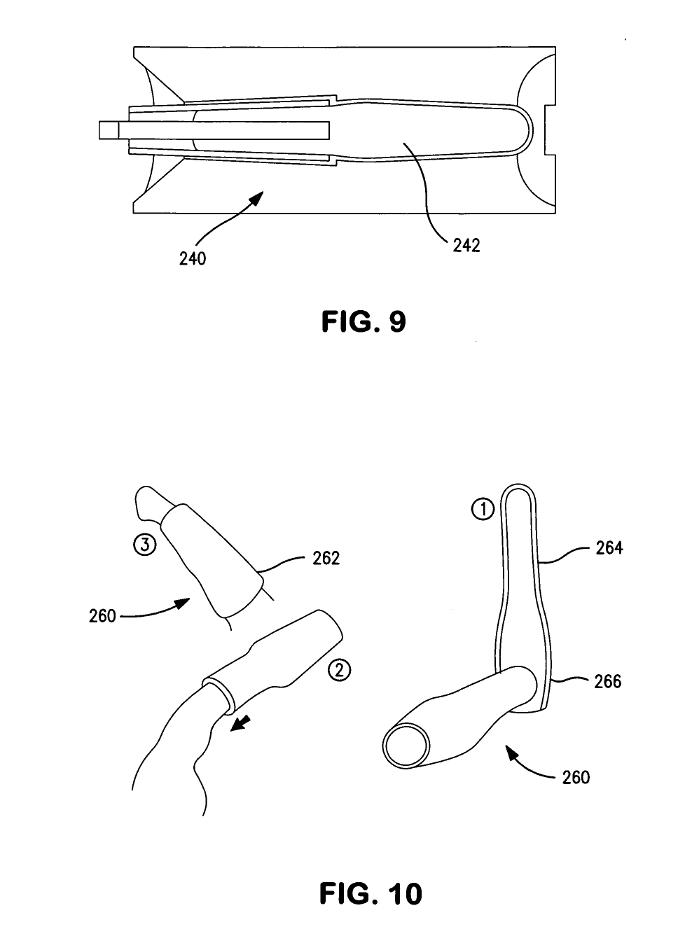

Referring to FIGS. 9-11, in further embodiments, splints 240, 260 may be provided. As shown in FIG. 9, splint 240 may include a lining 242 made of a non-stick material, such as a fabric or a non-stick coating such as oil, powder or another lubricant for preventing any sticking of the splint if the splint is made of a material that has a tendency to stick during placement of the splint on a user's finger (see FIGS. 5B-5D). When a fabric lining is used, the lining may be stretched over the core of the mold, and the splint material would be poured over the lining.

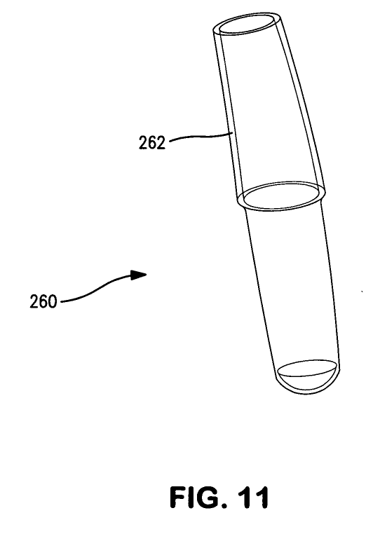

Referring to FIGS. 10 and 11, for splint 260, instead of using stays as discussed above with reference to splint designs 100, 150, the material properties of the splint may be varied at various strategic locations. For example, splint 260 may be created in hyperextension, or include a thicker/less stretchy band over the dorsal side at 262 to introduce enough force to straighten the joint without stays. The splint wall thickness may also be varied along the length thereof at locations 264, 266.

To summarize, compared to existing splint designs, splints 100, 150, 240 and 260 provide superior fit, adherence and skin health, and predetermined distribution of pressure on the dorsal side of a joint, and visual monitoring of the skin health. The splint according to the invention also are donned in a tight manner and allow for stretching, which is especially useful for users with pain and swelling in their hands. The splint designs of the invention provide for adequate cushioning of the joint and distribute the load over the whole surface area, and further stay on with circumferential tension so that they does not roll and all the pressure is not focused only over the dorsal side of the joint. Each of these factors separately or together facilitate patient compliance. Further, those skilled in the art would readily appreciate in view of this disclosure that the splint designs of the invention may be formed of a variety of sizes and shapes, and the wall thickness along the inner or outer layers may be varied without departing from the scope of the invention.

Although the exemplary embodiments of the splint have been described to treat joint contracture, those skilled in the art would appreciate the additional applications of the splint without departing from the scope of this invention. For instance the splint could be used to treat Mallet Finger, which is a condition where the extensor tendon of the finger is damaged and results in flexion of the distal interphalangeal (DIP) joint. Treatment for this condition is performed hyper-extending the DIP joint for an extended period of time until the tendon heals, which is possible by applying the splint with the appropriate shape.

Similarly, those skilled in the art can appreciate that the invention could be used to address DIP contractures in addition to the PIP contractures through subtle changes in the shape of the splint to focus treatment on the DIP joint.

Although the exemplary conditions treated by the splint have been orthopedic, those skilled in the art would appreciate that the invention could address neuromuscular disorders such as paralysis brought on by a cerebral vascular accident, or other reasons such as a traumatic injury. For instance, paralysis in certain muscles of the hand can result in flexor forces at wrist and digits which are often great due to tone, spasticity, contractures. The splint could be used to supplement the extensors (working against the flexors) so that the hand does not naturally go into a claw posture.

The invention described here could also be used to treat injuries of the joints while athletes participate in events. Specifically, rules in sporting events often may prohibit use of stiff materials such as rigid or semi-rigid plastics or metals to treat injuries because it could lead to additional injuries during the events. Those skilled in the art would appreciate that one of the virtues of the splint invention is that it can provide treatment to a joint either to extend, flex, or stabilize the joint without employing stiff material.

Although several embodiments of this invention have been described above with a certain degree of particularity, those skilled in the art may make numerous alterations to the disclosed embodiments without departing from the scope of this invention. All directional references (e.g., upper, lower, upward, downward, left, right, leftward, rightward, top, bottom, above, below, vertical, horizontal, clockwise and counterclockwise) are only used for identification purposes to aid the readers understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not as limiting. Changes in detail or structure may be made without departing from the invention as defined in the appended claims.

We claim:

1. A splint for treatment of a joint, the splint comprising:
a generally longitudinal body including first and second portions forming inner and outer splint layers, the second portion being rollable onto the first portion to form the splint.

2. A splint according to claim 1, wherein the outer splint layer includes at least one generally longitudinal cavity for insertion of a stay for adding rigidity to the splint structure.

3. A splint according to claim 2, further comprising a plurality of cavities disposed in one of a random and symmetrical pattern around the splint body.

4. A splint according to claim 1, wherein the outer splint layer includes at least one fluted section for permitting insertion of a stay between the inner and outer splint layers for adding rigidity to the splint structure.

5. A splint according to claim 1, wherein the generally longitudinal body is generally linear.

6. A splint according to claim 1, wherein the generally longitudinal body is generally curved.

7. A splint according to claim 1, wherein the generally longitudinal body is generally curved in a predetermined direction so as to place a predetermined torque on a joint when the splint is donned.

8. A splint according to claim 1, wherein at least one of the first and second portions include areas having different thicknesses for adding rigidity to the splint structure at a predetermined location.

9. A splint according to claim 1, further comprising a crease integrally formed along a length of the splint for facilitating predetermined donning of the splint.

10. A splint according to claim 1, wherein the splint is used for treatment of contracture of a proximal interphalangeal joint of a hand.

11. A splint according to claim 1, further comprising at least one cutout for exposing a predetermined portion of a user's finger.

12. A splint according to claim 1, wherein the splint is made of a flexible material.

13. A splint according to claim 1, wherein the splint is made of at least one of rubber, silicone and urethane.

14. A splint according to claim 1, further comprising a reduced friction outer surface layer for minimizing sticking of the splint during donning.

15. A splint according to claim 14, wherein the reduced friction outer surface layer includes at least one of a fabric, a lubricant and a powder layer.
